(19) United States
(12) Patent Application Publication
(10) Pub. No.: US 2022/0192347 A1
(43) Pub. Date: Jun. 23, 2022

(54) METHOD AND DEVICE FOR ANTI-AGING OF SKIN

(71) Applicant: Shiseido Company, Ltd., Chuo-ku, Tokyo (JP)

(72) Inventors: Daigo INOUE, Tokyo (JP); Satoshi HORIBA, Tokyo (JP); Atsushi SOGABE, Tokyo (JP); Kazuki TAKAGAKI, Tokyo (JP); Mieko NASU, Tokyo (JP); Tsuyoshi OHTANI, Tokyo (JP); Tomoya UCHIYAMA, Tokyo (JP); Akiko TAKAHASHI, Tokyo (JP)

(73) Assignee: Shiseido Company, Ltd., Chuo-ku, Tokyo (JP)

(21) Appl. No.: 17/603,869

(22) PCT Filed: Apr. 20, 2020
(86) PCT No.: PCT/JP2020/017099

§ 371 Date: Oct. 14, 2021

(30) Foreign Application Priority Data
Apr. 19, 2019 (JP) 2019-080490
Jun. 11, 2019 (JP) 2019-108921

(51) Int. Cl. A45D 44/22

(52) U.S. Cl. CPC A45D 44/22

(57) ABSTRACT

[Problem] To provide a method and device for anti-aging of skin. The present invention relates to a method and device for anti-aging of skin, comprising application of physical stimulation to the skin of the subject at a vibrational frequency of 60 Hz or lower.

[Solution] There are provided a method and device that exhibit a desirable skin anti-aging effect onto the shallow layers such as the epidermis.

### FIELD

The present invention relates to a method and device for anti-aging of skin, and particularly to a method and device for anti-aging of skin wherein skin is subjected to physical stimulation with a vibrational frequency of 60 Hz or lower.

### BACKGROUND

Skin aging is the alteration of skin caused by numerous factors including internal factors such as aging, hormones and metabolism, and external factors such as ultraviolet rays and drying. Skin aging can usually be judged based on exterior appearances such as skin spots, wrinkles, sagging and shrinkage, but aging also proceeds by non-visible changes in the interior skin tissue structure and its constituent components. Because these facets of aging usually proceed without being noticed, they are difficult to prevent. It is important to take appropriate measures to prevent and ameliorate skin aging.

A large number of cosmetic methods currently exist. PTL 1, for example, discloses an electroactive material actuator that causes displacement of keratin substances. PTL 2 discloses a photoirradiating cosmetic tool that exposes skin to photoirradiation while it is in a stretched state. PTL 3 discloses a slimming method for subcutaneous fat reduction, that combines stretching in which subcutaneous fat cells are stretched, with application of an external preparation for skin comprising a component that inhibits fat accumulation and/or a component that inhibits differentiation of adipocytes. PTL 4 discloses a stretching stimulation-mediated collagen-degrading enzyme production inhibitor that comprises an extract from an oat plant or an extract from a Psoralea corylifoli plant. PTL 5 discloses a stretching stimulation-induced wrinkle ameliorator or NF-κB activation inhibitor. NPL 1 recommends mechanical stimulation at 65 to 85 Hz. PTL 6 is related to the device described in NPL 1, and discloses a terminal effector that stimulates skin at a frequency of about 65 to about 120 Hz.

When stimulation is applied from the skin surface, however, the stimulation first affects the epidermis at the top surface, and can therefore cause epidermal damage. The basal membrane which has a highly characteristic structure (an undulating structure) is also present directly below the epidermis, and it is also thought to undergo the effects of mechanical stimulation. Prior art publications have in fact reported that physical stimulation does significantly alter the surface layers (NPL 2). It is therefore necessary to provide a method and device with a desirable anti-aging effect on skin, including the epidermis and basal membrane.

### CITATION LIST

### Patent Literature

[PTL 1] Japanese Patent Public Inspection No. 2011-505897
[PTL 2] Japanese Unexamined Patent Publication No. 2009-28271
[PTL 3] Japanese Unexamined Patent Publication No. 2015-127315
[PTL 4] Japanese Unexamined Patent Publication No. 2011-162507
[PTL 5] Japanese Unexamined Patent Publication No. 2014-108924
[PTL 6] Japanese Patent Public Inspection No. 2018-501,000

### Non Patent Literature

[NPL 1] Caberlotto et al. PLoS One. 2017 Mar. 1; 12
[NPL 2] Naruse et al. PLoS One. 2015 Nov. 3; 10
[NPL 3] Furukawa et al., 2017, Cell Reports 20, 1435-1447
[NPL 4] Cell Death and Disease (2014) 5, e1519; doi:10.1038/cddis.2014.476
[NPL 5] Current Biology Vol 24 No 17, 8 Sep. 2014, Pages 2012-2017
[NPL 6] Nature Communications volume 8, Article number: 15206 (2017)
[NPL 7] Development 138, 3907-3914 (2011) doi:10.1242/dev.070987
[NPL 8] Cell Reports 19, 1495-1502, May 23, 2017
[NPL 9] Nature Communications volume 9, Article number: 2961 (2018); DOI: 10.1038/s41467-018-05388-x
[NPL 10] Nature Cell Biology volume 20, pages262-271 (2018)
[NPL 11] Trends Cell Biol. 2018 July; 28(7):560-573. doi: 10.1016/j.tcb.2018.03.001
[NPL 12] Nature Communications volume 8, Article number: 15206 (2017; DOI: 10.1038/ncomms15206
[NPL 13] Cancer Res; 73(12) Jun. 15, 2013; DOI: 10.1158/0008-5472.CAN-12-3793

### SUMMARY

### Technical Problem

It is an object of the present invention to provide a method and device for anti-aging of skin.

### Solution to Problem

As a result of intensive research, the present inventors have completed this invention upon finding that when skin is subjected to physical stimulation such as stretching with a specific stretching rate and vibrational frequency, a desirable skin anti-aging effect is exhibited in the shallow layers such as the epidermis.

The present application provides an invention with the aspects set forth below.

(1) A cosmetic method for anti-aging of skin of a subject, which includes a process of applying weak physical stimulation to skin, the process comprising application of physical stimulation to the skin of the subject in which a cycle that includes, for example:

(a) stretching the skin of the subject to a stretching rate of 0.1% to 50.0%; and

(b) restoring the skin of the subject from the stretched state; and/or

(a-1) pressing the skin of the subject by 1 μm to 1000 μm; and

(b-1) restoring the skin of the subject from the pressed state;

is carried out at a vibrational frequency of 60 Hz or lower, where the stretching rate is calculated by:

Stretching rate (%)=(Distance from fixed point A to fixed point B after application of physical stimulation−distance from fixed point A to fixed point B before application of physical stimulation)/distance from fixed point A to fixed point B before application of physical stimulation×100 (Formula 1)

(where fixed points A and B are any arbitrary locations on the epidermis or a matrix attached to the epidermis, a straight line passing through fixed points A and B being parallel to the stretching direction).

(2) The cosmetic method according to (1), wherein one or more sets are carried out, where a set is defined as being 2 to 500 cycles.

(3) The cosmetic method according to (1) or (2), wherein when the percentage of the number of cells with nuclear-localized YAP with respect to the total number of keratinocytes is measured as an index, the skin anti-aging is defined as an increased percentage thereof measured in stretched keratinocytes after the application of the cosmetic method to skin as compared with the corresponding percentage before the application of the cosmetic method, where the percentage of the number of cells with nuclear-localized YAP is calculated by:

Percentage of number of cells with nuclear-localized YAP (%)=Number of cells with nuclear-localized YAP/total number of keratinocytes×100 (Formula 2).

(4) The cosmetic method according to (1) or (2), wherein when the percentage of the number of cells with actin stress fibers with respect to the total number of keratinocytes is measured as an index, the skin anti-aging is defined as an increased percentage thereof measured in stretched keratinocytes after application of the cosmetic method to skin, where the percentage of the number of cells with actin stress fibers is calculated by:

Percentage of number of cells with actin stress fibers (%)=Number of cells with actin stress fibers/total number of keratinocytes×100 (Formula 3).

(5) A cosmetic device for anti-aging of skin,

wherein the device comprises:

a stimulation generator that produces physical stimulation, and

a stimulation applicator that applies the physical stimulation generated by the stimulation generator to skin,

the device serving to carry out a process in which weak physical stimulation is applied to skin, and

the process carries out a cycle that includes, for example:

(a) stretching the skin to a stretching rate of 0.1% to 50.0%; and

(b) restoring the skin of the subject from the stretched state; and/or

(a-1) pressing the skin of the subject by 1 μm to 1000 μm; and

(b-1) restoring the skin of the subject from the pressed state;

at a vibrational frequency of 60 Hz or lower,

where the stretching rate is calculated by Formula 1 above.

(6) A cosmetic method for anti-aging of skin that includes application of the cosmetic device according to (5) to skin of a subject.

(7) The cosmetic method according to any one of (1) to (4) and (6), which is for increasing hyaluronic acid in skin.

(8) A cosmetic counseling method, for supporting cosmetology, which includes providing a cosmetic method according to any one of (1) to (4), (6) and (7) or a cosmetic device according to (5) to a subject.

### Advantageous Effects of Invention

According to the invention there are provided a method and device that exhibit a desirable skin anti-aging effect onto the shallow layers such as the epidermis. It is therefore expected to prevent and ameliorate aspects of skin aging such as skin spots, wrinkles and sagging.

### BRIEF DESCRIPTION OF DRAWINGS

FIG. 1 shows setting of stretching stimulation for Experiments 1 and 3.

FIG. 2a shows localization of YAP (red) and actin (green) without stretching stimulation and immediately after stretching stimulation in a monolayer culture of keratinocytes obtained from a 44-yr-old donor in Experiment 1.

FIG. 2b shows localization of YAP (red) and actin (green) without stretching stimulation and immediately after stretching stimulation in a monolayer culture of keratinocytes obtained from donors of respective different ages (0 years old, 18 years old and 64 years old) in Experiment 1.

FIG. 3 shows the percentage of the number of cells with nuclear-localized YAP without stretching stimulation and immediately after stretching stimulation in cultured keratinocytes obtained from donors of different ages (0 years old, 18 years old and 62 years old) in Experiment 1. In the graph, * indicates the significant difference based on a two-tailed t-test (equal variance of two parent populations) (p<0.05%).

FIG. 4 shows the percentage of the number of cells with actin stress fibers, without stretching stimulation and immediately after stretching stimulation, in cultured keratinocytes obtained from donors of respective different ages (0 years old, 18 years old and 62 years old) in Experiment 1. In the drawings, * indicates the significant difference based on a two-tailed t-test (equal variance of two parent populations) (p<0.05%).

FIG. 5 shows localization of YAP (red), actin (green) and nuclei (blue) without stretching stimulation and immediately after stretching stimulation in cultured fibroblasts obtained from donors of respective different ages (0 years old and 68 years old) in Experiment 1.

FIG. 6 shows the device used in Experiment 2, with an ex vivo skin sample.

FIG. 7 shows setting of stretching stimulation for Experiments 2, 4 and 5.

FIG. 8 shows localization of YAP (green) and nuclei (blue) without stretching stimulation and immediately after pressing stimulation of a skin sample obtained from a 44-yr-old donor in Experiment 2.

FIG. 9 shows localization of YAP and BrdU under conditions without stretching stimulation in monolayer cultures of keratinocytes obtained from a 44-yr-old donor in Experiment 3, under conditions of applying stretching stimulation once and applying stretching stimulation twice, followed by one week of normal culturing.

FIG. 10 shows the device used in Experiment 4, with a skin sample.

FIG. 11 shows localization of YAP (green), Ki67 (red) and nuclei (blue) under conditions with and without stretching stimulation once on two skin samples (sample #1 and #2) obtained from a 44-yr-old donor in Experiment 4, followed by one week of normal culturing.

FIG. 12 shows localization of YAP (green), Ki67 (red) and nuclei (blue) under conditions with and without stretching stimulation twice on two skin samples (sample #1 and #2) obtained from a 44-yr-old donor in Experiment 4, followed by one week of normal culturing.

FIG. 13 shows localization of hyaluronic acid (green), collagen (red) and nuclei (blue), under conditions with and without stretching stimulation twice on two skin samples (sample #1 and #2) obtained from a 44-yr-old donor in Experiment 5, followed by one week of normal culturing.

FIG. 14 shows an example of a device of the invention.

FIG. 15a shows measurement results using a VapoMeter and a Comeometer in Experiment 6. The “*” symbol represents significant difference with the value on day 0 by Dunnett's test (***: p<0.005, **: p<0.01, *: p<0.05).

FIG. 15b shows RO values obtained with a Cutometer in Experiment 6. The “*” symbol represents significant difference with the value on day 0 by Dunnett's test (***: p<0.005, **: p<0.01, *: p<0.05).

FIG. 15c shows the results of subjective evaluation in Experiment 6. The “*” symbol represents significant difference with the value on day 0 by Wilcoxon signed-rank test (***: p<0.005, **: p<0.01, *: p<0.05).

FIG. 16 (top left) shows the setting for Experiment 7. FIG. 16 (top right) shows an example of the waveform (square wave) for pressing stimulation in Experiment 7. FIG. 16 (bottom) shows the conditions for pressing stimulation in Experiment 7 (waveform: Shape of wave, vibrational frequency: Frequency/Hz, depth: Displacement/μm, period: Days).

FIG. 17 shows localization of Ki67 (green) and nuclei (blue) on day 1 (d1) and day 5 (d5), under conditions without pressing stimulation (Cont.) and with square wave pressing stimulation (0.5 Hz/100 μm), on two skin samples (sample #1 and #2) obtained from a 44-yr-old donor in Experiment 7.

FIG. 18 shows the percentage of the number of cells exhibiting Ki67 positivity from among the total number of cells on day 1 (left) and day 5 (right), under conditions without (Cont) and with (square wave: 0.5 Hz, 5 Hz, 50 Hz/100 μm) pressing stimulation once, on a skin sample obtained from a 44-yr-old donor in Experiment 7.

FIG. 19 shows the settings for Experiment 8 (top) and the waveform of pressing stimulation (square wave) applied (bottom).

FIG. 20a shows the results for Experiment 8, on day 0 and day 7, for pressing stimulation at left, and no pressing stimulation (control) at right. FIG. 20a represents the measurement results using a Corneometer.

FIG. 20b shows the results for Experiment 8, on day 0 and day 7, for pressing stimulation at left, and no pressing stimulation (control) at right. FIG. 20b represents the measurement results using a VapoMeter.

FIG. 20c shows the results for Experiment 8, on day 0 and day 7, for no pressing stimulation (control) at left, and pressing stimulation at right. FIG. 20c represents the RO value obtained using a Cutometer. The “*” symbol represents significant difference with the value on day 0 by Dunnett's test (*: p<0.05).

### DESCRIPTION OF EMBODIMENTS

A large variety of cosmetic methods currently exist that target the dermis or deeper layers, and they include strengthening of facial expression muscles, acceleration of fat decomposition and reinforcement of dermal fibers. Cosmetic methods focused on keratin substances such as in PTL 1 also exist, but most rely on electrical stimulation or strong stimulation by application of high vibrational frequencies. However, applying stimulation from the skin surface as described above can cause damage to the epidermis. Moreover, stretching stimulation such as described in PTLs 4 and 5 is also known to adversely affect the skin, and in fact it has been reported in NPL 2 that the surface layer is significantly altered by stretching stimulation.

The present inventors therefore conducted diligent research focused on the skin surface layer. As a result, it was found that aging of keratinocytes is associated with the behavior of YAP and the structure of actin fibers. For example, when skin was subjected to physical stimulation such as stretching, the percentage of cells with nuclear-localized YAP and the number of cells with actin stress fibers notably decreased in the case of aged keratinocytes. The present inventors further found that YAP behavior and actin fiber structure can be used as an index of skin aging, and that application of specific physical stimulation to the skin of a subject exhibits a desirable effect for skin including the epidermis. More specifically, even with aged epidermis, it was suggested that application of physical stimulation at a specific stretching rate with a vibrational frequency of 60 Hz or lower, such as 1 Hz or lower or 10 Hz or lower, has an effect of suppressing the reduction in the percentage of the number of cells with nuclear-localized YAP or actin stress fibers that is characteristic of aged keratinocytes. Such physical stimulation was also found to increase the amount of hyaluronic acid not only in the epidermis but also in the dermis, supporting its effectiveness for prevention and amelioration of skin aging.

The present invention provides a method for anti-aging of skin of a subject which includes applying physical stimulation to skin of a subject. The invention further provides a device for anti-aging of skin, comprising a stimulation generator that produces physical stimulation, and a stimulation applicator that applies the physical stimulation generated by the stimulation generator to skin.

The method of the invention comprises a process of applying weak physical stimulation to skin, and the device of the invention is a device for carrying out the process of applying weak physical stimulation to skin, the process including carrying out a cycle that includes, for example, (a) stretching skin to a stretching rate of 0.1% to 50.0%; and (b) restoring it from the stretched state, at a vibrational frequency of 60 Hz or lower.

The stretching rate is calculated by Formula 1 above. The physical stimulation may be carried out to a stretching rate of 0.001% to 80.0%, 0.01% to 60.0% or 0.1% to 50.0%, and preferably 0.1% to 50.0%. A stretching rate in any arbitrary range may be employed, such as 0.1% to 1.0%, 0.1% to 5.0%, 0.1% to 10.0%, 0.1% to 20.0%, 0.1% to 30.0%, 1.0% to 5.0%, 1.0% to 10.0%, 1.0% to 20.0%, 1.0% to 30.0%, 1.0% to 50.0%, 10.0% to 20.0% or 10.0% to 30.0%, for example.

The stretching speed is the speed (%/s) at which the maximum stretching rate (%) is reached during one cycle. The recovery speed is the speed (%/s) at which it returns to a non-stretched state from the maximum stretching rate. The stretching speed and recovery speed may be any arbitrary speed such as 0.010%/s to 40%/s, 0.05%/s to 30%/s, 0.10%/s to 20%/s, 0.2%/s to 15%/s or 0.3%/s to 10%/s. The stretching speed and recovery speed may be the same or different.

The method of the invention comprises a process of applying weak physical stimulation to skin, and the device of the invention is a device for carrying out the process of applying weak physical stimulation to skin, the process including carrying out a cycle that includes, for example, (a-1) pressing the skin of a subject by 1 μm to 1000 μm; and (b-1) restoring the skin of the subject from the pressed state; at a vibrational frequency of 60 Hz or lower.

The phrase “pressing the skin by 1 μm to 1000 μm” means that the skin is pressed to a depth of 1 μm to 1000 μm from the outer surface of the skin. The depth of compression may be set as desired, such as 1 μm to 1000 μm, 10 μm to 1000 μm, 10 μm to 300 μm or 10 μm to 100 μm from the outer surface of the skin.

The vibrational frequency is the number of cycles per second, where one cycle is a cycle from the start of stretching or compression to recovery to a non-stretched or non-pressed state. One cycle may also include maintaining a stretched or pressed state for a fixed time period, and/or pausing in a non-stretched or non-pressed state. For example, one cycle may also include (a′) maintaining a stretched or pressed state for 0 seconds to 30 minutes, 1 second to 20 minutes, 5 seconds to 10 minutes or 10 seconds to 5 minutes, either after (a) and before (b), or after (a-1) and before (b-1); and/or (b′) pausing at the non-stretched or non-pressed state for 0 seconds to 30 seconds, 0 seconds to 20 seconds, 0 seconds to 10 seconds, 1 second to 10 seconds, 1 second to 20 seconds or 1 second to 10 seconds, ether after (b) and before (a) in the next cycle, or after (a-1) and before (b-1). The vibrational frequency may be 0.0000001 Hz to 10 kHz, 0.000001 Hz to 1 kHz or 0.00001 Hz to 100 Hz, and is preferably 0.0001 Hz to 60 Hz or 0.0001 Hz to 10 Hz. For example, a vibrational frequency in any arbitrary range such as 0.001 Hz to 60 Hz, 0.01 Hz to 60 Hz, 0.001 Hz to 10 Hz, 0.01 Hz to 10 Hz, 0.1 Hz to 60 Hz, 0.1 Hz to 10 Hz, 0.5 Hz to 60 Hz, 0.5 Hz to 50 Hz, 0.5 Hz to 10 Hz, 0.5 Hz to 5 Hz, 0.5 Hz to 1 Hz, 0.001 Hz to 0.01 Hz, 0.001 Hz to 0.1 Hz, 0.001 Hz to 1 Hz, 0.01 Hz to 1 Hz, 0.1 Hz to 1 Hz, 1 Hz to 60 Hz, 1 Hz to 10 Hz or 1 Hz to 5 Hz may be employed.

Commercially available facial massagers and other massage equipment use electromagnetic waves with a frequency of about 0.3 to 300 MHz (such as RF waves), or ultrasonic waves with a frequency of about 1 MHz to 7 MHz. The vibrational frequency used by the method/device of the invention is extremely low compared to these frequency/vibrational frequency ranges. When a strong vibrational frequency is applied to skin as with a conventional facial massager, it can potentially produce adverse effects on skin such as redness, pressure marks, scratches, pain or inflammation, but application in the vibrational frequency range of the invention reduces this risk and allows noninvasive physical stimulation. The present inventors have found that an excessively high stretching rate, degree of compression or vibrational frequency causes overly strong stimulation, and that it is therefore preferred to adjust the values to appropriate ranges to stimulate skin in a more gentle manner.

It has been technical knowledge to those skilled in the art that with the types of cosmetic devices conventionally used in the field, which employ common motor mechanisms, it is only possible to select vibrational frequencies above 60 Hz due to the mechanical mechanisms of the motors. Construction of special machinery has been required to employ vibrational frequencies in the range of the present invention, such as 60 Hz or lower, 10 Hz or lower or 1 Hz or lower, which are vibrational frequencies of “60 Hz or lower”, considered to be the limit for cosmetic equipment in the prior art. In addition, it has been firmly accepted that such low vibrational frequencies are “too low” to exhibit the effect of the invention, for which reason almost no research has been done in this regard. The present inventors nevertheless attempted to actually subject skin to physical stimulation using vibrational frequencies that are extremely low in terms of the common technical knowledge of the prior art and found, surprisingly, that a satisfactory effect was exhibited even with gentle stimulation at such low vibrational frequencies.

While low-to-medium frequency devices are commercially available, such as EMS devices, they are especially designed to act in the deep layers such as muscle or subcutaneous fat, and it is unclear what effects they have on the skin surface layer, as according to the present invention. Such devices often produce prickly stimulation even when a low-frequency current flows, and therefore differ from the present invention which applies more gentle stimulation to skin. The present invention can provide a simple cosmetic method of applying stretching or pressing stimulation directly to the skin, without application of energy such as ultrasonic waves, electrical current or a magnetic field. Stretching or pressing stimulation at the specified vibrational frequency, while being gentle stimulation, also exhibits a satisfactory effect on dermal cells in addition to epidermal cells, as described in the Examples. Using the method and device of the invention is therefore expected to provide a cosmetic effect on skin epidermis over short periods and on the dermis over long periods, without adversely affecting the skin.

Physical stimulation may be massaging using an instrument such as a facial massager, or an experimental device, or a human hand or instrument, or facial exercising, and it may be either with or without contact. According to one aspect, mechanically generated physical stimulation can be applied to skin using a device that comprises a stimulation generator that produces physical stimulation, and a stimulation applicator that applies the physical stimulation either with or without contact. The physical stimulation may be carried out by contact, such as tension, compression, tapping, pinching or suction of the skin, and/or by non-contact such as causing displacement by application of shock waves to the skin using ultrasonic waves or air pressure, for example. Exercising of the face may be inflation of the cheeks or wide opening of the eyes. Massaging may be performed using the hand of the subject undergoing the operation or of an operator such as a cosmetologist, or using an instrument such as a roller. This is not limitative, however, so long as it is within the scope of physical stimulation according to the invention.

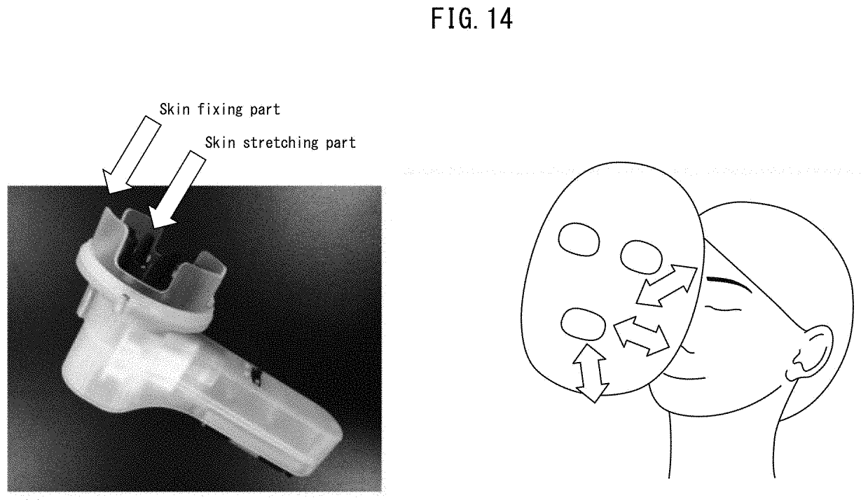

For example, the device of the invention may be a cosmetic apparatus comprising a skin-contacting part that contacts with the skin of the user to apply physical stimulation according to the invention. It may also comprise a gripping part and a skin stretching part or skin pressing part. For example, the device shown at left in FIG. 14 is designed so that the skin-contacting part contacts with skin, thus stretching the skin with a specific vibrational frequency and stretching rate.

Alternatively, for example, the device of the invention may comprise a power source, a stimulation generator and a skin stimulating part, the power source generating an electrical signal, the stimulation generator converting the electrical signal from the power source to physical stimulation and providing the physical stimulation, and the skin stimulating part receiving the physical stimulation generated by the stimulation generator and applying the physical stimulation to the skin of the user.

The device shown at left in FIG. 14, for example, comprises a gripping part, a power source, a controller that controls the physical stimulation, a stimulation generator, and a skin-contacting part that includes a skin stimulating part and a skin fixing part. The design is such that the user holds the gripping part with the skin-contacting part against the skin and fixes the skin with the skin fixing part, operating the controller to convert electrical signals from the power source to physical stimulation by the stimulation generator, with the physical stimulation being transmitted to the skin stimulating part and causing the skin to be stretched at the specified vibrational frequency and stretching rate by the skin stimulating part while it is fixed by the skin fixing part. For example, the stimulation generator may be driven by a motor to convert electrical signals to physical stimulation. The skin stimulating part shown at left in FIG. 14 applies stretching stimulation to skin, but it may also apply pressing stimulation to skin.

Alternatively, the device of the invention may be a cosmetic apparatus comprising a power source, a controller that controls physical stimulation, a stimulation generator, and a skin-contacting part that includes a skin contact surface made of a sheet-like material. An example is the skin-contacting part of the cosmetic apparatus shown at right in FIG. 14. The sheet-like material may be one that allows flow of current to convert electrical signals from a power source into physical stimulation. Such sheet-like materials include dielectric elastomer actuators (DEA), conductive polymers, IPMC, PVC gels and McKinnen-type materials.

The power source of the device of the invention may be an internal power source or external power source, and may also be rechargeable. The device of the invention may use data stored in a cellular phone or cloud, for example, and may be remotely operated in a wireless manner.

The physical stimulation may be such as to cause stretching or compression of the skin by application of the physical stimulation in a contact or non-contact manner as described above. The physical stimulation may be applied parallel to the skin surface, i.e. horizontally, or perpendicular to the skin surface, i.e. vertically, or in any other direction such as diagonally or in a twisted direction.

The number of cycles for the physical stimulation is not restricted. Any number of cycles such as 2 to 500 cycles, 10 to 500 cycles, 20 to 400 cycles, 30 to 300 cycles, 40 to 200 cycles or 50 to 100 cycles may be carried out, for example. It will often be sufficient to carry out 27 cycles as described in the Examples.

With an arbitrary number of cycles as 1 set, an arbitrary number of sets, such as 1 to 100 sets, 2 to 50 sets or 3 to 10 sets may be carried out.

The time period for the physical stimulation is also not restricted. For example, the cycles may be repeated, with or without a pause period, for a fixed period of 5 minutes to 3 hours, 10 minutes to 2 hours or 30 minutes to 1 hour.

The time interval between cycles or between sets is not restricted. For example, stretching or pressing stimulation may be carried out in a single set or multiple sets, with the one or more sets being carried out once every day, or every 2 days, 3 days, 4 days, 5 days, 6 days or 7 days, or once every week or every 2, 3 or 4 weeks, either continuously or intermittently, and regularly or irregularly.

However, the vibrational frequency, stretching rate, number of cycles and frequency of use are not restricted so long as sufficient stimulation is provided to exhibit an effect of skin anti-aging. The waveform for physical stimulation may also be set as desired, as a square wave, sine wave, triangular wave or sawtooth wave, for example.

The subject to which the method of the invention is applied may be a subject having objective or subjective skin aging, or a subject desiring to prevent skin aging. For example, it may be a subject judged to be deficient in constituent components of skin, such as hyaluronic acid. According to one embodiment, the subject may be one judged to have a high degree of skin aging, using YAP behavior and/or actin fiber structure in keratinocytes as the index. For example, the subject may be one who is judged to have a low percentage of the number of cells with nuclear-localized YAP with respect to the total number of keratinocytes, and/or a low percentage of the number of cells with actin stress fibers with respect to the total number of keratinocytes, based on measurement of stretched keratinocytes. Alternatively, the subject may be one who is concerned about signs of skin aging such as skin spots, wrinkles, sagging, loss of color clarity and visibility of pores, or reduced smoothness, firmness, tension, luster and elasticity. Skin spots, wrinkles, sagging, loss of color clarity and visibility of pores, or reduced smoothness, firmness, tension, luster and elasticity, can be determined using publicly known indexes, such as visual assessment, subjective or objective evaluation, or Cutometer values.

Skin anti-aging may also be determined by measuring the percentage of the number of keratinocytes with nuclear-localized YAP with respect to the total number of keratinocytes. The presence or absence of nuclear-localized YAP can be determined as described in the Examples, by staining endogenous YAP in the cells, observing and photographing endogenous YAP in the nucleus or cytoplasm using a microscope, and using image processing analysis software for digitization of the ratio between nuclear-localized YAP and cytoplasmic YAP based on fluorescence intensity. Alternatively, it can be measured by the method described in NPL 6 or NPL 7. The percentage of the number of keratinocytes with nuclear-localized YAP with respect to the total number of keratinocytes is calculated by Formula 2 above. For example, the number of cells found to have nuclear-localized YAP by the method described above may be counted, and the cells with nuclei stained by nuclear staining using DAPI or Hoechst33342 may be counted as the total number of cells, for calculation according to Formula 2.

According to one embodiment, “nuclear localization of YAP” means that the increasing or decreasing total expression level of endogenous YAP in keratinocytes is in a range of no greater than twice the amount before and after the application, where YAP in the nuclei functions as a transcriptional factor. This is because it has been confirmed, based on NPLs 3 to 5, that the (protein) expression level is not significantly accelerated even when YAP inhibitors have been deleted to promote nuclear localization of YAP (YAP protein level is times on average in NPL 5, for example).

YAP is Yes-associated protein, an approximately 65 kDa protein also known as YAP1 or YAP65. It has been reported that YAP functions as a transcriptional regulatory factor, that it is involved in cell proliferation and apoptosis, and that it is associated with maintenance and establishment of stem cells (niches) and in tumor or cancer occurrence. YAP is degraded in the cytoplasm, but upon localizing to the nucleus it becomes activated and regulates transcription. Localization of YAP has also been reported to be associated with extracellular matrix hardness, cell density, and cell proliferation and self-replication (NPLs 11 to 13).

Formation of stress fibers in the cytoplasm consisting of the YAP upstream factor actin is known to be correlated and positively associated with nuclear localization of YAP. Skin anti-aging according to the invention, therefore, may also be determined using the presence or absence of actin stress fibers in keratinocytes as an index. For example, skin anti-aging can be determined by measuring the percentage of the number of keratinocytes with actin stress fibers with respect to the total number of keratinocytes. The presence or absence of actin stress fibers, as shown in the Examples, can be determined by whether or not actin stress fibers are present, when the actin fibers are stained with fluorophore conjugated with phalloidin and at least one actin stress fiber is present in the cytoplasm or around the nucleus. Alternatively, it can be measured by the method described in NPL 9 or NPL 10. The percentage of the number of keratinocytes with actin stress fibers with respect to the total number of keratinocytes is calculated by Formula 3 above. For example, the number of cells found to have actin stress fibers may be counted by the method described above, and the total number of cells may be counted by DAPI staining, for example, and calculation performed using Formula 3.

According to one embodiment, keratinocytes with actin stress fibers are keratinocytes in which actin stress fibers have been formed without increase in actin protein expression level. This is because the amount of actin protein does not vary even with difference in the presence or absence of YAP or the localization of the YAP (nuclear or cytoplasmic), as taught in NPL 8.

Therefore, the occurrence of skin anti-aging in the present invention may also be indicated by a suppressed reduction in the percentage of the number of cells with nuclear-localized YAP or actin stress fibers with respect to the total number of keratinocytes among keratinocytes after applying stretching stimulation to a skin sample, by using the method or apparatus of the present invention. The suppression may be, for example, suppression in reduction of the percentage, with a statistically significant difference (in a Student's t test, for example) at a significance level of 5%, and/or suppression of 10% or greater, 20% or greater, 30% or greater, 40% or greater, 50% or greater, 60% or greater, 70% or greater, 80% or greater, 90% or greater or 100%, for example.

Nuclear-localized YAP and actin stress fibers are known to function indirectly or directly in cell proliferation and self-replication (NPL 11). On the other hand, it has become clear that YAP outside the nucleus or cytoplasmic YAP, degradation of cytoplasmic YAP, or lack of actin stress fiber formation, is associated with mechanisms of elimination from the cell population by apoptosis or differentiation (NPL 12). Findings from research on skin keratinocytes and various other cell types suggest that keratinocytes without localization of YAP in the nucleus, or without actin stress fiber formation, have low growth-maintenance and self-replicating ability, and become senescent or differentiated cells (NPL 13). In the Examples described herein, the number of aged keratinocytes with weak nuclear-localized YAP retention or weak actin stress fiber formation was significantly reduced by the method/device of the invention. Therefore, without being constrained by theory, this suggests that the method/device of the invention selectively eliminates aged keratinocytes, promoting selection of “young” keratinocytes that have nuclear-localized YAP retention or actin stress fiber formation, and their subsequent reactivation and proliferation. In other words, as a result of selectively eliminating keratinocytes without localization of YAP in the nuclei or without actin stress fiber formation, and selectively leaving keratinocytes with localization of YAP in the nuclei and keratinocytes with actin stress fibers, in keratinocytes stretched by applying specific physical stimulation to skin according to the invention, it raises a possibility that that the invention causes growth or self-replication of the keratinocytes. In other words, the method/device of the invention applies physical stimulation to selectively eliminate aging keratinocytes and select keratinocytes that proliferate and self-replicate, thus potentially resulting in increased proliferation and restoration of keratinocytes.

During measurement of the number of cells with nuclear-localized YAP or actin stress fibers, it is preferred to apply physical stimulation such as stretching stimulation to a skin sample. The physical stimulation used during measurement of skin aging or a skin anti-aging effect is not limited and may be the same as or different from physical stimulation used in the anti-aging method/device of the invention, if it is sufficient stimulation for measurement of the degree of skin aging. For example, using a skin sample that is skin in the body, harvested skin, or cultured keratinocytes, the skin sample can be stretched by applying physical stimulation through contact by tension, compression, tapping, pinching or suction, and/or non-contact by displacement using shock waves on the skin with ultrasonic waves or air pressure, with no limitation on the direction of applying the physical stimulation.

Either alternatively and/or additionally, the skin anti-aging effect may also be an increase in the amount of hyaluronic acid in the dermis by the method/device of the invention. The increase may be, for example, increase in hyaluronic acid, with a statistically significant difference (in a Student's t test, for example) at a significance level of 5%, and/or increase of 10% or greater, 20% or greater, 30% or greater, 40% or greater, 50% or greater, 60% or greater, 70% or greater, 80% or greater, 90% or greater or 100% or greater.

Either alternatively and/or additionally, the skin anti-aging effect may be an increase in signs such as skin viscoelasticity, firmness, tension, luster, smoothness, visibility of pores and lack of unclear spots, by the method/device of the invention. Skin viscoelasticity, firmness, tension, luster and smoothness can be measured using a Cutometer, or based on a subjective or objective index. The increase may be, for example, increase with a statistically significant difference (in a Dunnett's test or Wilcoxon signed-rank test, for example) at a significance level of 5%, and/or increase of 10% or greater, 20% or greater, 30% or greater, 40% or greater, 50% or greater, 60% or greater, 70% or greater, 80% or greater, 90% or greater or 100% or greater.

The method and device of the invention can prevent and ameliorate skin aging while exhibiting a desirable effect in the shallow layers such as the epidermis. For example, it is possible to improve the behavior of YAP or the structure of actin fibers in keratinocytes, and especially to suppress reduction, or to restore subsequent increase, in the percentage of number of cells with nuclear-localized YAP and actin stress fibers with respect to the total keratinocytes when physical stimulation has been applied to skin. It is also possible to cause increase in hyaluronic acid in the dermis, and also increase in skin viscoelasticity and the aforementioned subjective or objective indexes.

The method/device for anti-aging of skin according to the invention may also be used in combination with other treatments. Examples of such other treatments include, but are not limited to, light stimulation, electrical stimulation, mechanical stimulation, application of stimulation by the human hand, administration of anti-aging agents, or coating of cosmetics.

Anti-aging agents may be natural or chemically synthesized compounds, or extracts from an animal or plant, either alone or as mixtures, or contained in a solvent as a water-soluble solution, or in the form of a cosmetic. The route of administration of an anti-aging agent may be selected as desired, with examples including oral administration, transdermal administration, subcutaneous administration, transmucosal administration and intramuscular administration.

The method/device for anti-aging of skin according to the invention may be a method/device for cosmetic use, medical care, stress alleviation or relaxation. According to one embodiment, the method/device for anti-aging of skin of the invention is for cosmetic use, instead of being a treatment method or device used by a doctor or health care professional. The invention further provides cosmetic counseling method for supporting cosmetology, which includes providing a cosmetic method or device of the invention to a subject.

All of the publications referred to throughout the present specification are incorporated herein in their entirety in references.

### EXAMPLES

The present invention will now be explained in greater detail by examples. The examples of the invention are intended to serve merely as illustration and do not limit the technical scope of the invention. The technical scope of the invention is limited solely by the description in the Claims. Modifications of the invention, such as additions, deletions or substitutions to the constituent features of the invention, are possible so long as the gist of the invention is maintained.

### Experiment 1: Changes in Keratinocytes In Vitro Due to Aging

Samples: Cultures of monolayer cultured keratinocytes and fibroblasts from donors of different ages (0 years old, 18 years old, 44 years old and 64 years old) purchased from KAC Co., Ltd. were used.

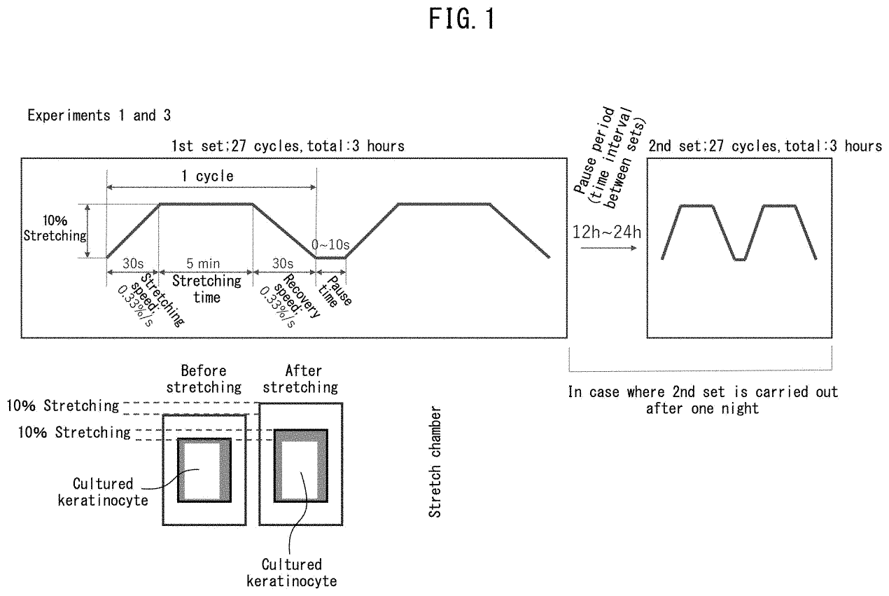

Stretching conditions: ShellPa by Menicon Life Science was used to apply stretching stimulation to the keratinocytes and fibroblasts. The stretching stimulation was carried out to a stretching rate of 10% as shown at center bottom in FIG. 1. One cycle was pulling the culture chamber in one direction at a stretching speed of 0.33%/s, for stretching of the cells to a stretching rate of 10%, as shown at left top in FIG. 1. The stretched state was then held for 5 minutes, and the cells were returned to the original non-stretched state at a recovery speed of 0.33%/s. With this procedure as one cycle, a total of 27 cycles were carried out over a period of 3 hours. The pause period between cycles was 0 to 10 seconds.

Observation method: Endogenous YAP with or without stretching stimulation was observed by fluorescent immunostaining method by using the following antibodies and fluorophore. YAP in the cells was detected using anti-YAP antibody (Santa Cruz Biotechnology, YAP antibody (63.7): sc-101199), with fluorophore conjugated with phalloidin (ThermoFisher Scientific, Alexa Fluor™ 488 Phalloidin: A12379) as the actin fibers, and observation and imaging were carried out using a confocal microscopy (Zeiss, LSM700). The image analysis software ImageJ was also used for digitization of the nucleus/cytoplasm ratio of YAP, based on fluorescence intensity. The nucleus/cytoplasm fluorescence intensity ratio (nuclear YAP fluorescence intensity/cytoplasmic YAP fluorescence intensity) was calculated, and the number of cells with values of was counted as cells with nuclear YAP. The total number of cells was determined by counting the cells whose nuclei were stained using DAPI (Vector Laboratories, VECTASHIELD Mounting Medium with DAPI:H-1200), and the percentage of the number of cells with nuclear-localized YAP was calculated by Formula 2. Cells containing even one actin stress fiber in the cytoplasm or around the nucleus were counted as actin stress fiber-containing cells, and the total number of cells was counted in the same manner and the percentage of the number of cells with actin stress fibers was calculated by Formula 3.

Results:

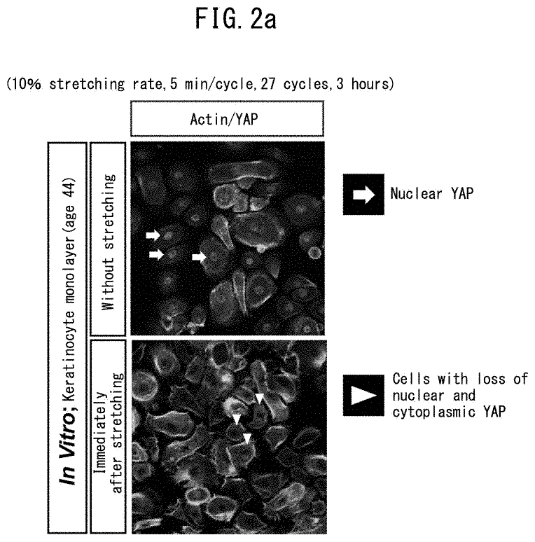

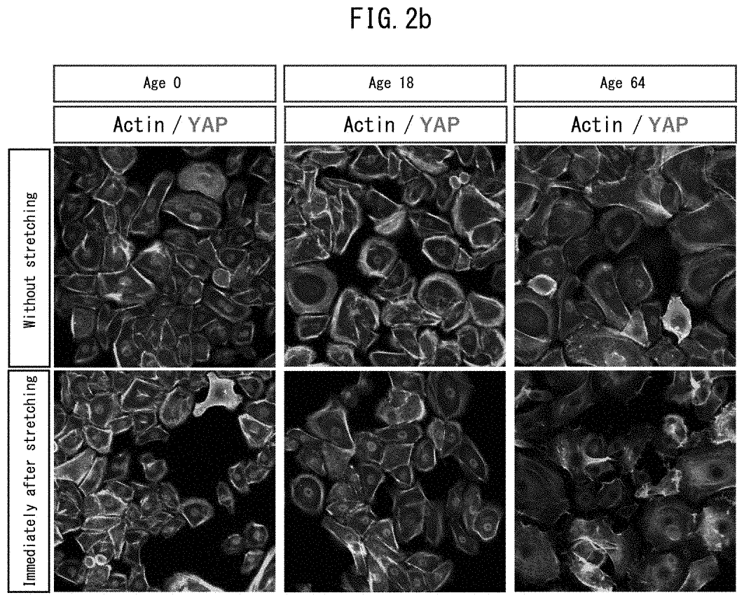

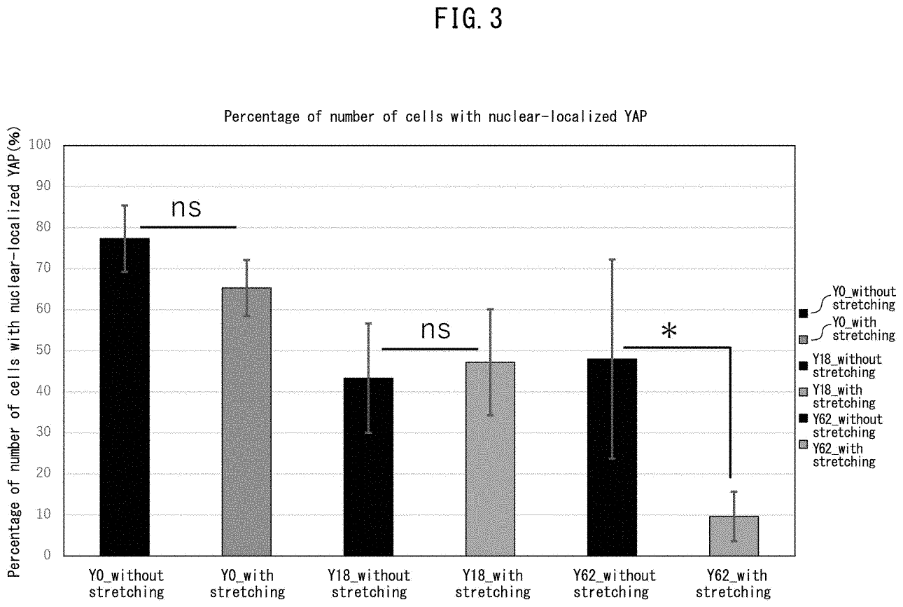

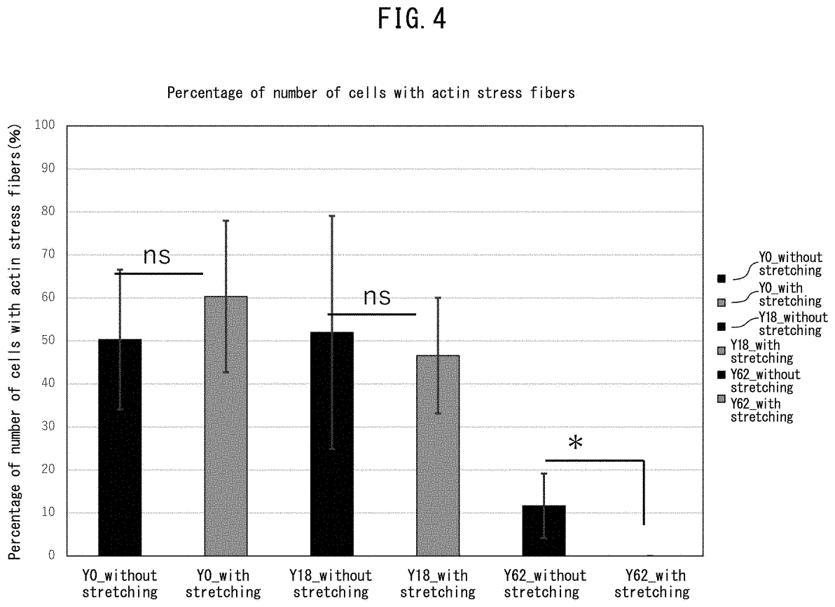

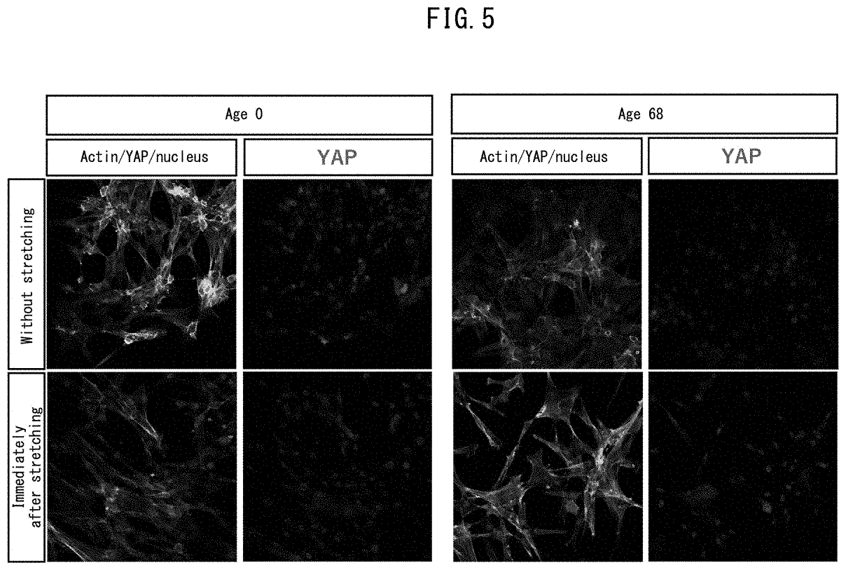

FIGS. 2a and 2b show photomicrographs of keratinocytes. As seen in the photographs, the keratinocytes obtained from the younger donors (age 0, 18) had nuclear YAP in almost all of the cells under both conditions with and without stretching stimulation, whereas the keratinocytes obtained from the middle-aged donors (age 44, 64) had a drastically reduced number of cells with nuclear-localized YAP upon application of stretching stimulation. FIG. 3 shows the percentages of number of cells with nuclear-localized YAP in keratinocytes. No change was seen in the number of cells with nuclear-localized YAP in the younger donors, but a significant reduction was observed in the elderly keratinocytes. Despite the sharp reduction, however, the number of cells with nuclear-localized YAP still did not fall to zero but remained at a fixed percentage. In FIG. 4 which shows the percentages of number of cells with actin stress fibers among keratinocytes, no change is seen in the number of cells with actin stress fibers among the younger donors, while there was significant reduction and almost disappearance in the keratinocytes among the elderly donors. In fibroblasts, as shown in FIG. 5, no change was seen in YAP or actin stress fibers under both conditions with and without stretching stimulation, with virtually no difference observed between the age groups.

### Experiment 2: Changes of Keratinocytes Ex Vivo Due to Aging

Samples: ex vivo human abdominal region skin tissue slices obtained from Genoskin Co. (NativeSkin, 12 well size, diameter: approximately 1 cm). The tissue slices were from a 38-year-old.

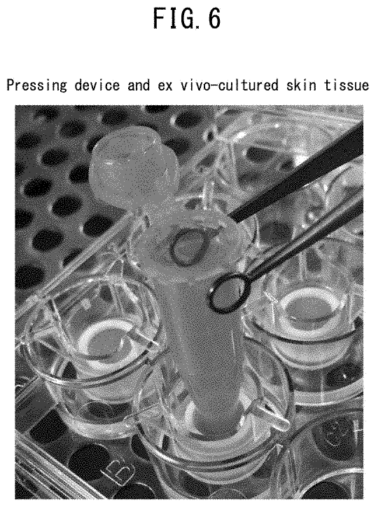

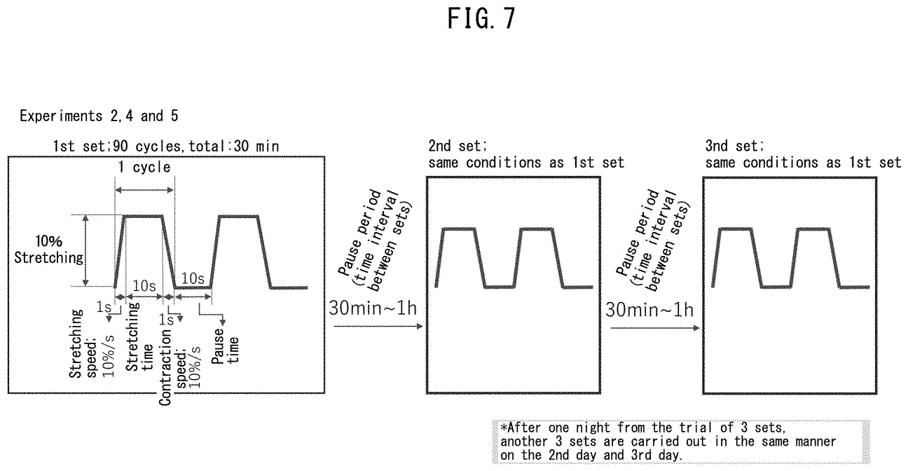

Stretching conditions: A plastic pressing part was constructed as shown in FIG. 6. The pressing part was moved down vertically from the top of the sample so that the bottom was pressed against the top surface of the sample, with adjustment for stretching of the skin to a stretching rate of 0.1% or lower. This pressing action was carried out at a speed of 10%/s as shown in FIG. 7, after which the pressing part was held and the stretched state was maintained for 10 seconds, the device was raised up at a speed of 10%/s to restore the original non-stretched state, and the procedure was paused for 10 seconds until the next pressing action. With this procedure as one cycle, a total of 90 cycles were carried out over a period of 30 minutes. With 90 cycles as one set, pressing action was carried out for a total of 3 sets (total of 270 cycles), with a pause period of 30 minutes to 1 hour between sets.

Observation method: YAP and DAPI were stained, observed and imaged by the same method as Experiment 1, under both conditions with and without stretching stimulation.

Results:

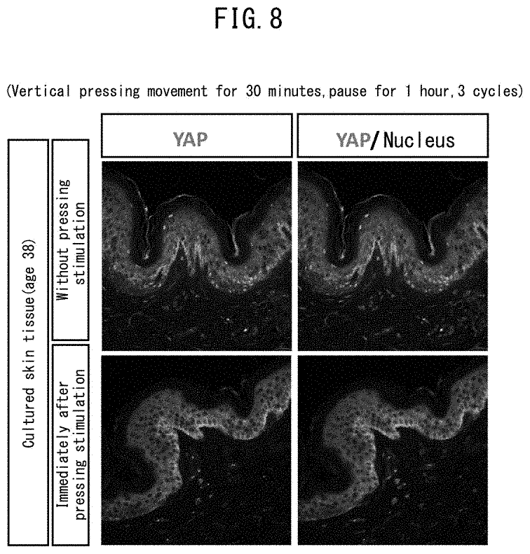

The change in YAP behavior due to aging seen in Experiment 1 was also seen in the ex vivo samples. As shown in FIG. 8, even in the ex vivo skin model obtained from a middle-aged donor (age 38), application of stretching stimulation in keratinocytes in the epidermis resulted in reduced nuclear-localized YAP and many non-stained areas. The change was particularly notable near the basal lamina.

### Experiment 3: Effect of Stretching Stimulation on Keratinocytes In Vitro

Sample: A monolayer culture of keratinocytes from a middle-aged donor (age 44) was used in the same manner as Experiment 1.

Stretching conditions: stretching stimulation was applied as in Experiment 1. Specifically, a set of the 27 cycles of stretching stimulation of Experiment 1 was applied. However, after 3 hours of one set of stretching stimulation comprising 27 cycles, and a lapse of 12 to 24 hours, the same stretching stimulation was again applied to the sample for a total of 2 sets.

Observation method: YAP was stained in the same manner as Experiment 1, without stretching stimulation and after stretching stimulation. The proliferation marker BrdU was also stained by fluorescent immunostaining method, using anti-BrdU antibody (Abcam, Anti-BrdU antibody [BU1/75 (ICR1)]: ab6326). BrdU treatment was carried out for 6 hours, and after 7 days, 4% paraformaldehyde was used for 24 hours of fixation at 4° C. The fixed sample was observed and imaged using a confocal microscopy (Zeiss, LSM700).

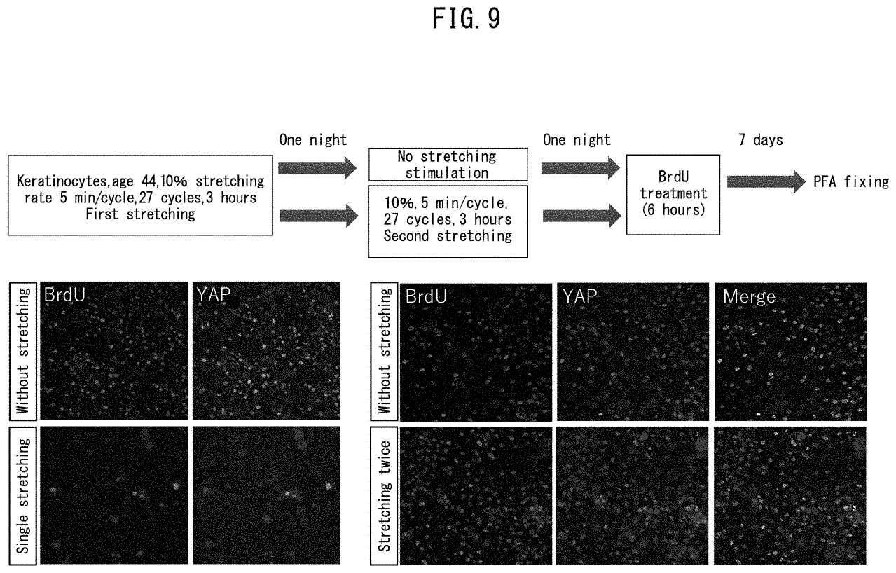

The results are shown in FIG. 9. In the first stretching stimulation, a significant change in the number of cells with nuclear-localized YAP was observed before and after stretching stimulation, similar to Experiment 1. BrdU was also drastically reduced by stretching stimulation, similar to nuclear-localized YAP. On the other hand, when second stretching stimulation was applied 24 hours after the first stretching stimulation, nuclear-localized YAP and BrdU were not found to significantly change, or were found to increase only slightly, between the conditions without stretching stimulation and the conditions with stretching stimulation applied twice. This suggested a correlation between nuclear-localized YAP and the proliferated cells, and suggested that the cells after application of the second stretching stimulation had increased numbers with auto-replicating ability compared to the first stretching stimulation, and had recovered or maintained cell growth ability similar to conditions without stretching, or young keratinocytes. Without being constrained by theory, it is thought possible that the first stretching stimulation, by some mechanism such as promoting apoptosis of aged keratinocytes that have low proliferation maintenance or self-replicating ability, or causing their detachment and shedding, may promote proliferation and restoration of keratinocytes by selectively excluding aged keratinocytes and selectively leaving cells with nuclear-localized YAP exhibiting proliferation maintenance or high self-replicating ability.

### Experiment 4: Effect of Stretching Stimulation on Epidermis Ex Vivo

Sample: An ex vivo human abdominal region skin tissue slice (NativeSkin, 6-well size, diameter: about 2 to 2.5 cm) of a middle-aged donor (age 42) purchased from Genoskin Co. was used.

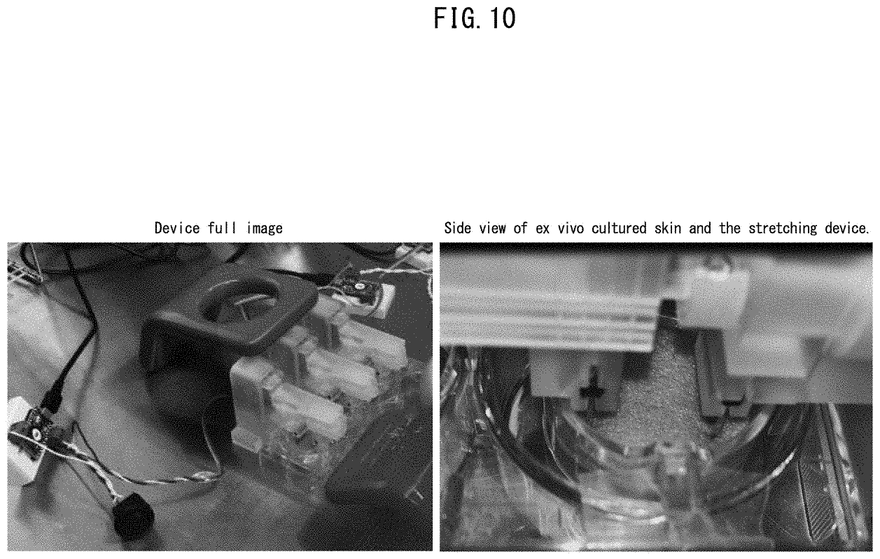

Stretching conditions: A stretching instrument was constructed, having a gripping part for gripping both ends of the skin in the wells, as shown in FIG. 10, and causing stretching of the skin by pulling of the gripping part. The wells containing the tissue strips were placed horizontal, the gripping part was operated to stretch the skin from both ends, and stretching was carried out at a speed of 10%/s to a 10% stretching rate as shown in FIG. 7, after which the samples were restored to their original non-stretched state at a recovery speed of 10%/s. With this procedure as one cycle, a total of 90 cycles were carried out over a period of 30 minutes. With 90 cycles as one set, a total of 3 sets (total of 270 cycles) were carried out, with a pause period of 30 minutes to 1 hour between sets. After 3 hours of 3 sets of stretching stimulation, and a lapse of 12 to 24 hours, the same 3 sets of stretching stimulation on the samples were carried out twice for 2 consecutive days.

Observation method: On day 0 without stretching stimulation, immediately after stretching stimulation (also day 0), and after one week of culturing the skin tissue without stretching stimulation and with stretching stimulation treatment, YAP and DAPI immunostainings were carried out in the same manner as Experiment 1, Ki67 was stained with anti-Ki67 antibody (Abcam, Anti-Ki67, antibody [SP6]: ab16667) as a proliferation marker, and observation and imaging were carried out by the same method as Experiment 1.

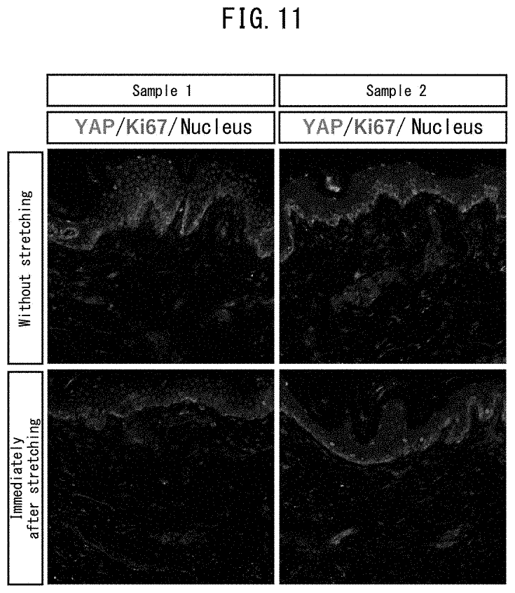

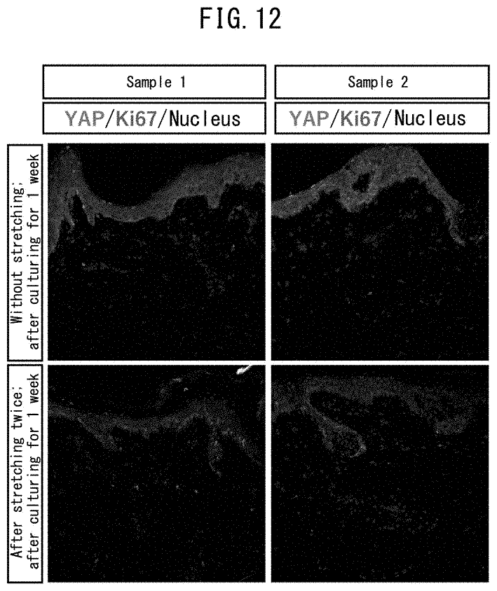

FIG. 11 shows the state on day 0 without stretching stimulation and immediately after stretching stimulation (also day 0). Epidermal cells in the basal layer with YAP were decreased, and the proliferation marker Ki67 increased immediately after tension. Without being constrained by theory, it is believed that stretching stimulation may have accelerated the course of cell proliferation and subsequent cell differentiation. FIG. 12 shows the state after culturing for one week after applying stretching stimulation twice on 2 consecutive days. Before stretching, YAP was seen not only in the basal lamina but also in all of the epidermal cells. YAP is usually abundant in the basal layer, but it was observed in all of the layers of the skin that had been cultured for one week. Without being constrained by theory, it is believed that in non-stretched skin tissue, cell senescence may result in some abnormality of cell proliferation or cell differentiation in each of the layers of the epidermis, causing variation in the distribution of YAP. However, the distribution of YAP expression in the basal layer was maintained after carrying out stretching stimulation twice. Without being constrained by theory, it is believed that physical stimulation may have activated and supported homeostasis of cell proliferation and cell differentiation in the epidermis, so that the YAP distribution supported the normal initial state of fresh skin tissue, even after culturing for one week.

### Experiment 5: Effect of Stretching Stimulation on Dermis Ex Vivo

Sample and stretching conditions: An ex vivo human abdominal region skin tissue slice (NativeSkin, 6-well size, diameter: about 2 to 2.5 cm) of a middle-aged donor (age 44) purchased from Genoskin Co. was used, similar to Experiment 4. Three sets of stretching stimulation, with stretching as in Experiment 4, were carried out twice on 2 consecutive days.

Observation method: The DAPI, hyaluronic acid (HOKUDO, Biotin-HABP: BC41) and collagen (Merck Millipore, Anti-human COL1N-terminal antibody: MAB1912) were observed before stretching stimulation and after culturing for one week after stretching stimulation. The DAPI was observed and imaged in the same manner as Experiment 1, and the hyaluronic acid and collagen were observed and imaged using a confocal microscopy (Zeiss, LSM700) with staining by fluorescent immunostaining method.

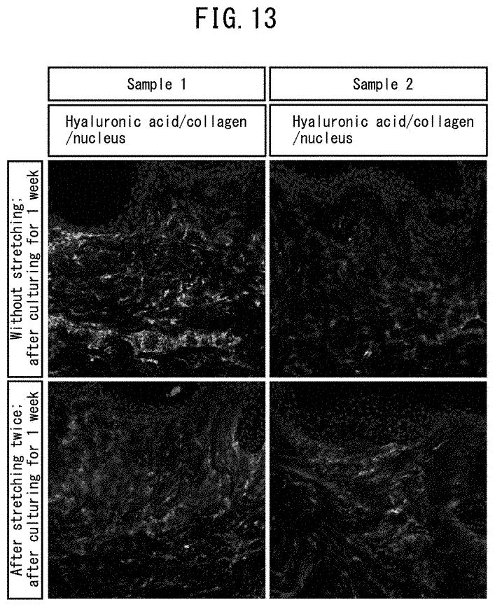

The results are shown in FIG. 13. When stretching stimulation was applied, hyaluronic acid increased in the dermis. Stretching stimulation was thus shown to have a desirable effect, not only for accelerating proliferation of epidermal cells but also on the dermis.

### Experiment 6: Effect of Stretching Stimulation In Vivo

Subjects: 17 healthy females aged 30 to 50 were selected.

Stretching conditions: The stretching device shown at left in FIG. 14 was used. This device is designed so that the skin-contacting part indicated by the arrows at left in FIG. 14 is contacted with the skin, so that the skin fixing part fixes the skin while appropriately setting the degree of indentation of the skin stretching part into the skin in the direction perpendicular to the skin to within a range with a maximum of about 5 mm, and with a horizontal stretching rate of 0.1 to 20%, with the skin being stretched at a vibrational frequency of 1.2 Hz. For 10 days, the subject carried out a procedure of applying the device to half of the face to apply stretching stimulation, for 30 minutes each day.

Measuring method: On day 3 and day 10 after start of the experiment, the condition of the skin on both halves of the face (with (treated) and without (control) stretching stimulation) was measured. For measurement of the skin condition, transepidermal water loss (TEWL) was measured using a VapoMeter by Delfin Technologies, skin moisture content was measured using a Corneometer (CM825) by Courage+Khazaka Co., and skin viscoelasticity was measured using a Cutometer (SEM575) by Courage+Khazaka Co. The obtained data were statistically analyzed by a Dunnett's test in comparison with the values at day 0 (***: p<0.005, **: p<0.01, *: p<0.05).

The participants subjectively evaluated their impression of the cheeks before applying stretching stimulation and 10 days afterwards, recording the evaluation on the following 5-level scale.

TABLE 1

Evaluation
Tension
Firnmess
Luster
No visible pores
No loss of skin clarity
Smooth
5-Level scale
5- Significantly changed
4- Changed
3- Indeterminable
2- No change
1 - Absolutely no change

The results of the subjective evaluation were statistically analyzed by a Wilcoxon signed-rank test (***: p<0.005, **: p<0.01, *: p<0.05).

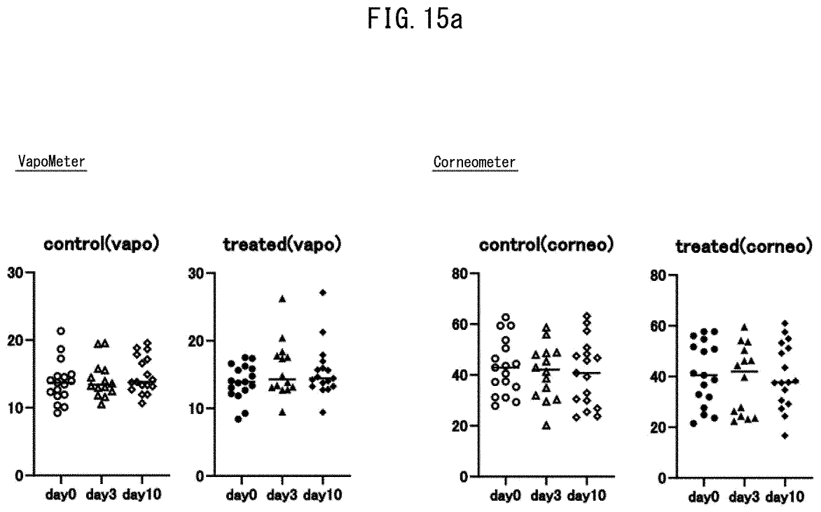

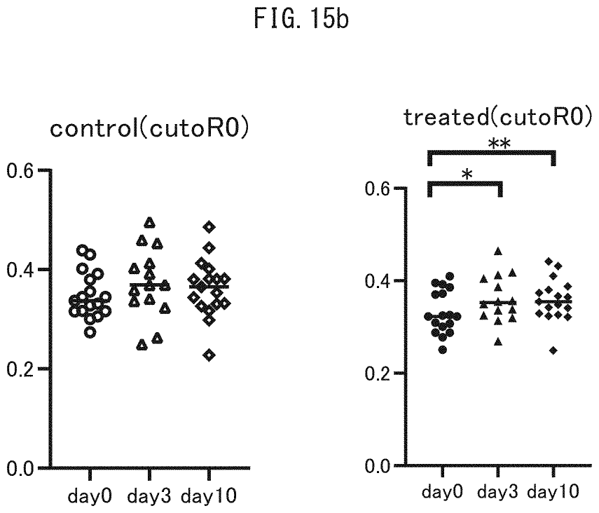

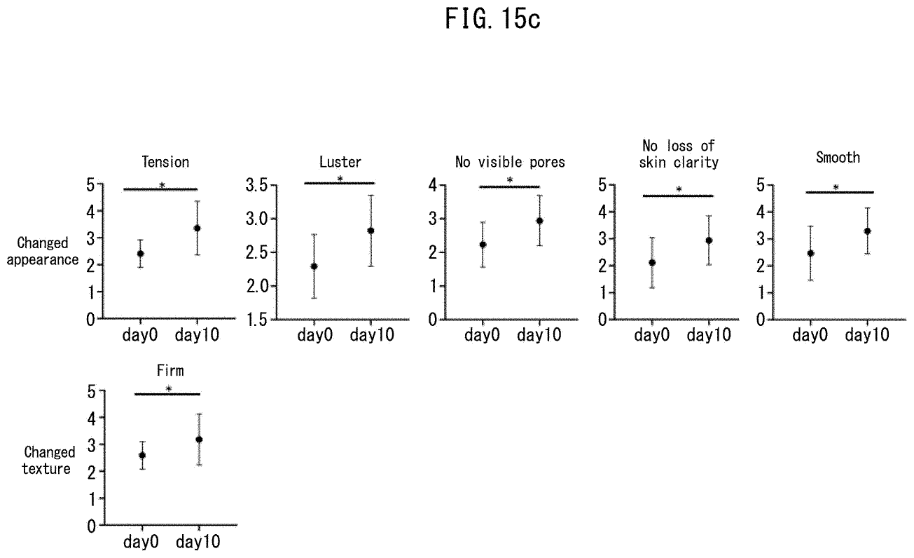

Results: The results are shown in FIGS. 15a to 15c. As shown in FIG. 15a, none of the TEWL or skin moisture contents were changed by stretching stimulation, but as shown in FIG. 15b, the RO value increased significantly based on Cutometer measurement, indicating improved skin viscoelasticity. The same tendency was seen for the R2 values and R7 values as well. As shown in FIG. 15c, the participants also judged the skin condition to have improved in terms of appearance and feel.

### Experiment 7: Effect of Pressing Stimulation Ex Vivo

Sample: An ex vivo human abdominal region skin tissue slice (NativeSkin, 6-well size, diameter: about 2 to 2.5 cm) of a middle-aged donor (age 44) purchased from Genoskin Co. was used, similar to Experiment 4.

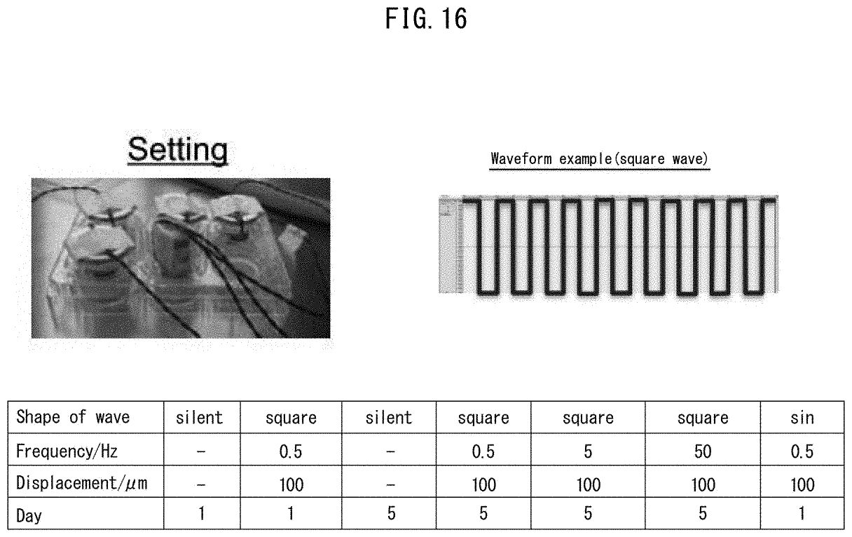

Compression conditions: A dielectric elastomer actuator (SRHP074-001 by Sumitomo Riko Co., Ltd.) was used. As shown at top left in FIG. 16, the dielectric elastomer actuator was used on a skin tissue slice, repeating contraction and expansion to apply pressing stimulation with different waveforms of 0 to 100 μm shown at bottom in FIG. 16, with a vibrational frequency of 0.5 Hz, 5 Hz or 50 Hz, for one day or 5 days.

Observation method: DAPI and Ki67 were observed by the same method as Experiment 4, before pressing stimulation and after pressing stimulation.

Results:

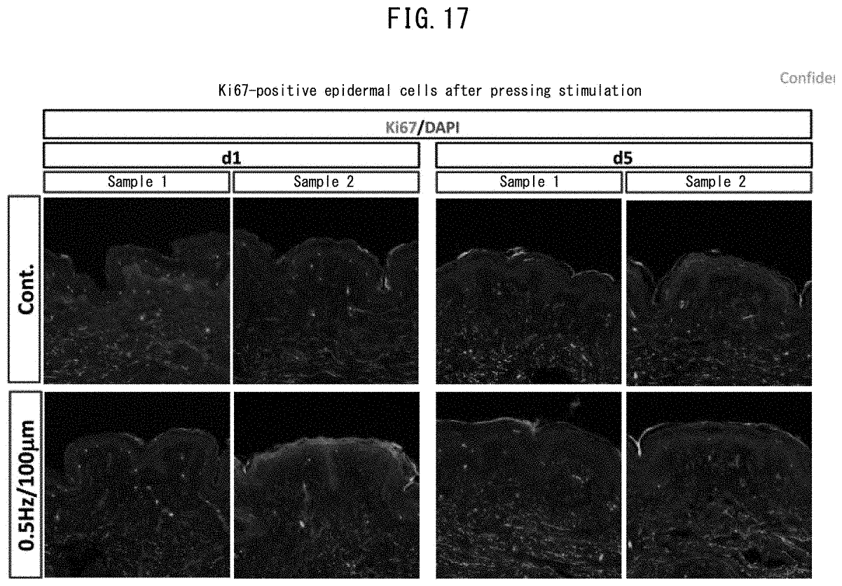

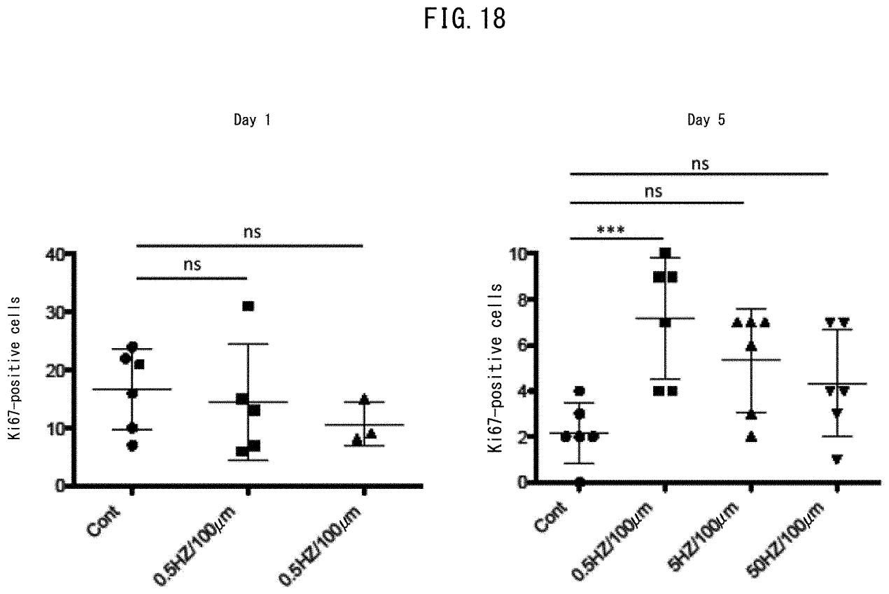

The results are shown in FIGS. 17 and 18. Without pressing stimulation (Cont: silent), Ki67-positive cells on the 5th day was decreased compared to the 1st day, but with pressing stimulation (0.5 Hz/100 μm: square), decrease in Ki67-positive cells in the epidermal cells on the 5th day was suppressed, indicating that pressing stimulation suppresses growth inhibition of epidermal cells. Compression stimulation with a low vibrational frequency of about 0.5 Hz exhibited a higher effect than 5 Hz or 50 Hz. The results shown in FIGS. 17 and 18 are with a square wave, but similar results were also obtained with a sine wave.

### Experiment 8: Effect of Pressing Stimulation In Vivo

Subjects: 17 healthy females aged 30 to 50 were selected.

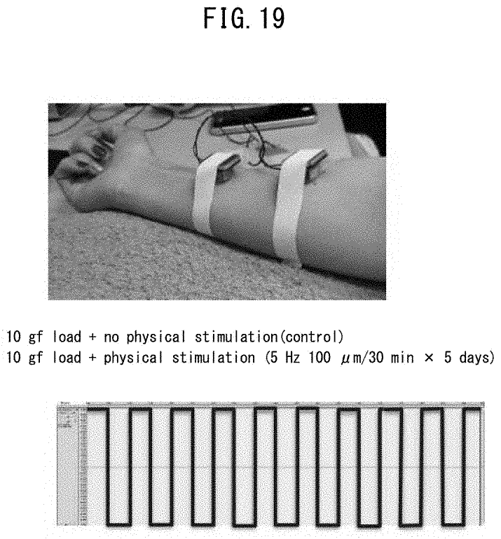

Stretching conditions: The compression device shown in FIG. 19, a dielectric elastomer actuator (SRHP074-001 by Sumitomo Riko), was used. The device is designed so that the skin-contacting part of the device shown at top in FIG. 19 pushes into the skin with a force of 10 gf, thus applying square wave pressing stimulation as shown at bottom in FIG. 19, with a vibrational frequency of 5 Hz to a depth of 100 μm. As shown in FIG. 19, the device was used on the inner forearm of the participant, for pressing stimulation 30 minutes every day for a period of 5 days.

Measuring method: The condition of the skin on the inner forearm, with and without application of pressing stimulation, was measured before start of the experiment and on the 7th day. For measurement of the skin condition, transepidermal water loss (TEWL) was measured using a VapoMeter by Delfin Technologies, skin moisture content was measured using a Corneometer (CM825) by Courage+Khazaka Co., and skin viscoelasticity was measured using a Cutometer (SEM575) by Courage+Khazaka Co.

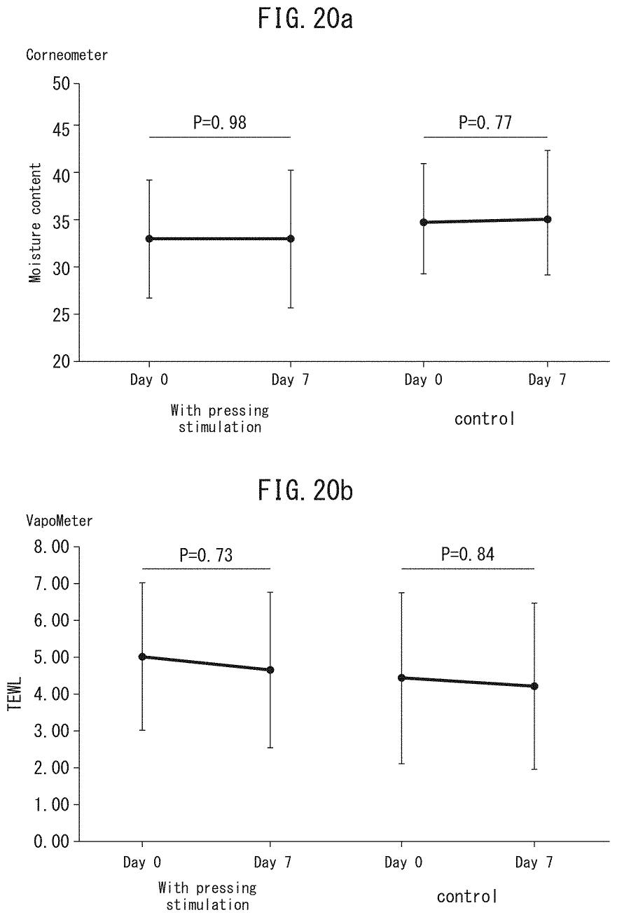

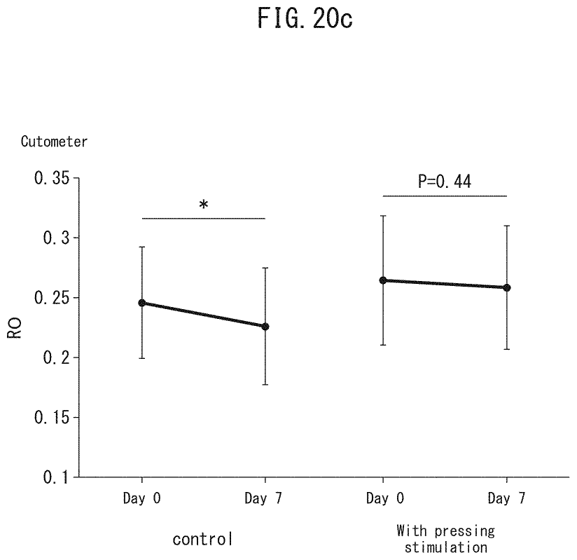

Results: The results are shown in FIGS. 20a to 20c. Both TEWL and skin moisture content were unchanged by pressing stimulation, but based on the Cutometer results, the RO value was shown to have significantly decreased on the 7th day with no pressing stimulation, while the decrease was suppressed and improved viscoelasticity was observed with pressing stimulation.

This suggested that in skin subjected to physical stimulation according to the invention, aged cells were selectively eliminated by stretching stimulation and keratinocytes with nuclear-localized YAP and actin stress fibers were selectively maintained. The results also suggested cell proliferation of the selected keratinocytes with nuclear-localized YAP and actin stress fibers. In addition, the level of hyaluronic acid had increased not only in the epidermis where keratinocytes were present, but also in the dermis. In other words, the results suggested that using the cosmetic method or device of the invention exhibits an anti-aging effect on skin, not only in the epidermis but also in the dermis. Using the cosmetic method or device of the invention on humans in vivo was also found in fact to improve the skin viscoelasticity, and to improve appearance and feel in terms of firmness, luster, loss of color clarity and smoothness.

1. A cosmetic method for anti-aging of skin of a subject, which includes a process of applying weak physical stimulation to skin, the process comprising application of physical stimulation to the skin of the subject in which a cycle that includes, for example:
(a) stretching the skin of the subject to a stretching rate of 0.1% to 50.0%; and
(b) restoring the skin of the subject from the stretched state; and/or
(a-1) pressing the skin of the subject by 1 μm to 1000 μM; and
(b-1) restoring the skin of the subject from the pressed state;
is carried out at a vibrational frequency of 60 Hz or lower, where the stretching rate is calculated by:
Stretching rate (%)=(Distance from fixed point A to fixed point B after application of physical stimulation−distance from fixed point A to fixed point B before application of physical stimulation)/distance from fixed point A to fixed point B before application of physical stimulation×100 (Formula 1)
(where fixed points A and B are any arbitrary locations on the epidermis or a matrix attached to the epidermis, a straight line passing through fixed points A and B being parallel to the stretching direction).

2. The cosmetic method according to claim 1, wherein one or more sets are carried out, where a set is defined as being 2 to 500 cycles.

3. The cosmetic method according to claim 1, wherein when the percentage of the number of cells with nuclear-localized YAP with respect to the total number of keratinocytes is measured as an index, the skin anti-aging is defined as an increased percentage thereof measured in stretched keratinocytes after the application of the cosmetic method to skin as compared with the corresponding percentage before the application of the cosmetic method, where the percentage of the number of cells with nuclear-localized YAP is calculated by:
Percentage of number of cells with nuclear-localized YAP (%)=Number of cells with nuclear-localized YAP/total number of keratinocytes×100 (Formula 2).

4. The cosmetic method according to claim 1, wherein when the percentage of the number of cells with actin stress fibers with respect to the total number of keratinocytes is measured as an index, the skin anti-aging is defined as an increased percentage thereof measured in stretched keratinocytes after application of the cosmetic method to skin, where the percentage of the number of cells with actin stress fibers is calculated by:
Percentage of number of cells with actin stress fibers (%)=Number of cells with actin stress fibers/total number of keratinocytes×100 (Formula 3).

5. A cosmetic device for anti-aging of skin,
wherein the device comprises:
a stimulation generator that produces physical stimulation, and
a stimulation applicator that applies the physical stimulation generated by the stimulation generator to skin,
the device serving to carry out a process in which weak physical stimulation is applied to skin, and
the process carries out a cycle that includes, for example:
(a) stretching the skin to a stretching rate of 0.1% to 50.0%; and
(b) restoring the skin of the subject from the stretched state; and/or
(a-1) pressing the skin of the subject by 1 μm to 1000 μM; and
(b-1) restoring the skin of the subject from the pressed state;
at a vibrational frequency of 60 Hz or lower,
where the stretching rate is calculated by Formula 1 above.

6. A cosmetic method for anti-aging of skin that includes application of the cosmetic device according to claim 5 to skin of a subject.

7. The cosmetic method according to claim 1, which is for increasing hyaluronic acid in skin.

8. A cosmetic counseling method, for supporting cosmetology, which includes providing a cosmetic method according to claim 1 to a subject.

9. A cosmetic counseling method, for supporting cosmetology, which includes providing a cosmetic device according to claim 5 to a subject.
